(19) United States
(12) Patent Application Publication
(10) Pub. No.: US 2012/0283747 A1
(43) Pub. Date: Nov. 8, 2012

(54) HUMAN-ROBOT SHARED CONTROL FOR ENDOSCOPIC ASSISTANT ROBOT

(75) Inventor: Aleksandra Popovic, New York, NY (US)

(73) Assignee: KONINKLIJKE PHILIPS ELECTRONICS N.V., EINDHOVEN (NL)

(21) Appl. No.: 13/509,330

(22) PCT Filed: Nov. 15, 2010
(86) PCT No.: PCT/IB10/55175

§ 371 Date: Jul. 30, 2012

Related U.S. Application Data
(60) Provisional application No. 61/261,390, filed on Nov. 16, 2009.

(51) Int. Cl. A61B 19/00

(52) U.S. Cl. 606/130

(57) ABSTRACT

A surgical system includes a robot with both an active mode and an inactive mode of operation, and a holding arm for holding a surgical tool, and an immediate deactivator for determining when a human operator manually manipulates a holding arm or a surgical tool depending on signals from at least one condition sensor. Immediately upon that determination, the immediate deactivator deactivates the robot. The holding arm includes a stiffener/destiffener for increasing or decreasing the flexibility of the holding arm. The stiffness of the holding arm can be sufficiently decreased in the inactive mode to allow a human operator to skillfully control repositioning the surgical tool into a new position while the flexible holding arm is connected between the robot and the surgical tool. Also, the stiffness of the holding arm can be sufficiently increased, for essentially locking it into a rigid fixed shape for providing sufficient rigidity in the active mode for the robot to reposition the rigid holding arm for repositioning the surgical tool to perform preprogrammed tasks initiated by surgeon command inputs. The holding arm is completely inactive in both the active and inactive modes of the robot.

The present invention generally relates to the field of robotic surgical systems and more specifically to robotic controllers and processes for controlling robotic surgical systems especially endoscopic robotic systems.

This application claims priority of U.S. provisional application Ser. No 61/261,390, filed Nov. 16, 2009, which is incorporated herein by reference.

An endoscope is an illuminated optic instrument for the visualization of the interior of a body cavity or organ. Typically, the endoscope is a long tube with a small video camera on the front end and a data cable trailing form the back end. The cable is attached to monitor that shows a magnified internal view of a surgical site. Instruments are available in varying lengths, diameters, and flexibilities. The fiberoptic endoscope has great flexibility, allowing it to reach previously inaccessible areas.

The endoscope may be introduced through a natural opening in the body or it may be inserted through an incision. Instruments for viewing specific areas of the body include the bronchoscope, cystoscope, gastroscope, laparoscope, otoscope, and vaginoscope. All of these scopes and similar scopes are referred to as endoscopes herein.

Endoscopy is the use of an endoscope during a surgical procedure. The purpose of endoscopy is to provide a minimally invasive surgery. In traditional surgery the body is opened primarily so that the surgeon can see the site that he is operating on. In minimally invasive surgery, rather than cutting patients open, endoscopy allows surgeons to operate through small incisions by allowing the surgeon to see the operating site using the endoscope. These less invasive procedures result in less trauma and pain for patients. Surgery through smaller incisions typically results in less scarring and faster recovery.

Robot-assisted surgery is the latest development in endoscopy. A robot arm is connected to the endoscope to hold in endoscope in position. The robot includes motors to move the robot arm to move the endoscope during surgery. The robot also includes a user input system for receiving commands from the surgeon to move the endoscope. The input system may include a microphone and voice recognition or a keyboard or a joystick or a mouse used with a graphical user interface. The robot also includes a controller to execute preprogrammed tasks to move the endoscope in response to the commands provided by the surgeon.

U.S. publication 2007/0142823 to Prisco et. al. discloses a robotic surgical system with a robot control system having both a normal mode and a clutch mode of operation. Buttons are used to switch between normal mode and clutch mode. In the normal mode the robot arms operate in a master/slave mode using input devices such as a joystick to guide robot arm movements. In the clutch mode, the robot arms can be directly manipulated by the surgeon by grasping the arms and moving them. In the clutch mode, a control system operates the motors of the robot arms to compensate for internally generated friction and inertial resistance to provide easy manipulation of the position of the robot arms.

EndoAssist (Prosurgics Ltd, UK) is an example of an endoscope assistant with master/slave architecture that is described in Sashi S. Kommu et al “Initial Experience With The Endoassist Camera-Holding Robot In Laparoscopic Urological Surgery”, J Robotic Surg (2007) 1:133-137. The surgeon controls the robot through head motion measured by head-mounted infrared sensor. In order to activate robot control, the surgeon needs to release a food pedal.

A non-robotic passive system Endofreeze (Aesculap, Germany) uses flexible passive arms for holding endoscopes, without an active component as described in A. Arezzo et al. “Experimental Assessment Of A New Mechanical Endoscopic Solosurgery System”. Surg Endosc (2005) 19: 581-588.

Kwon et al. Chapter 15: Intelligent Laparoscopic Assistant Robot Through Surgery Task Model: How To Give Intelligence To Medical Robots ISBN 978-3-902613-18-9, describes a shared-control system in which the robot is able to follow tools and perform similar automatic tasks, but the surgeon can take over control using speech-control and activation button/pedal.

In one aspect of the invention of this application, a surgical system includes a robot that has both an active mode of operation and an inactive mode of operation. In the active mode the robot controls the repositioning of a surgical tool, such as an endoscope, during a surgical procedure. In the inactive mode of operation, the robot is substantially immobile and rigid. The robot has a controller preprogrammed with predetermined tasks to perform during a surgical procedure. The surgical system includes a user input communicating with the controller for a user to initiate the execution of the preprogrammed tasks in the active mode;

The surgical system also includes an elongate holding arm with a first end and a second distal end. The first end has a connector for connection to the robot, the second distal end has a connector for connection to the surgical tool. The holding arm includes a stiffener/destiffener for increasing or decreasing the flexibility of the holding arm. The stiffness of the holding arm can be sufficiently decreased in the inactive mode to allow a human operator to skillfully control repositioning the surgical tool into a new position while the flexible holding arm is connected between the robot and the surgical tool. Also, the stiffness of the holding arm can be sufficiently increased, for essentially locking it into a rigid fixed shape for providing sufficient rigidity in the active mode for the robot to reposition the rigid holding arm for repositioning the surgical tool to perform the tasks. The holding arm is completely inactive in both the active and inactive modes of the robot.

A condition sensor on a robot arm and/or the holding arm and/or the surgical tool communicates with the controller for producing signals depending on a mechanical condition of the holding arm and/or surgical tool. The condition sensor may indicated (measure) the shape of a robot arm and/or the holding arm and/or the condition sensor may indicate (measure) the forces and/or moments on an arm of the robot and/or the holding arm and/or the surgical tool and/or the condition sensor may indicate (measure) the position of the robot arm and/or the holding arm and/or the surgical tool and/or the condition sensor may indicate that a user has grasped the holding arm and/or the surgical tool.

The surgical system also includes an immediate deactivator for determining when a human operator manually manipulates the holding arm and/or the surgical tool depending on signals from the condition sensor. Immediately upon that determination, the immediate deactivator deactivates the robot by changing the mode of operation of the robot from active mode to inactive mode.

reactivation means for reactivating the robot in response to user input means by changing the mode of operation of the robot from inactive mode to active mode in a current position of the surgical tool and for the robot to resume controlling the repositioning of the surgical tool during the surgical procedure.

In another aspect of the invention, in a surgical system, shape sensors are provided on a robot arm and/or on a inactive holding arm for indicating (measuring) the approximate shape of the robot arm and/or holding arm during the surgical procedure. The controller includes a shape predictor for predicting the shapes of the holding arm while performing tasks during the surgical procedure. The shape predictor calculates a theoretical shape. The immediate deactivation means deactivates the robot when the indicated shape deviates from the predicted shape according to a predetermined criteria for determining when the human operator manually manipulates the second end of the holding arm and/or the surgical tool.

In another aspect of the invention, a surgical system of claim 1, again shape sensors are provided on the robot arm and/or holding arm for indicating (measuring)the approximate shape of the robot arm and/or holding arm during a surgical procedure. Also, an initial shape of the flexible arm is determined when the robot is activated. The immediate deactivator deactivates the robot when the difference between the indicated shape and the initial shape exceeds a threshold for determining when the human operator is manually manipulating the second end of the holding arm and/or the surgical tool.

In another aspect of the invention, in a surgical system, a displacement sensor indicates (measures) an approximate linear and/or rotational displacement of the surgical tool and/or the distal end of the holding arm during the surgical procedure. A controller includes a displacement predictor for predicting linear and/or rotational displacements of the surgical tool and/or the distal end of the holding arm while performing tasks during the surgical procedure. The displacement predictor calculates a theoretical displacement. An immediate deactivator deactivates the robot when the indicated displacement deviates from the predicted displacement according to a predetermined criteria for determining when the human operator manually manipulates the second end of the holding arm and/or the surgical tool.

In another aspect of the invention, in a surgical system, a displacement sensor indicates (measures) an approximate linear and/or rotational displacement of the surgical tool and/or the distal end of the holding arm during the surgical procedure. An initial linear and/or rotational displacement of the surgical tool and/or the distal end of the holding arm is determined when the robot is activated. An immediate deactivator immediately deactivates the robot when the difference between the indicated linear and/or rotational displacement and the initial linear and/or rotational displacement exceeds a threshold for determining that the human operator is manually manipulating the second end of the holding arm and/or the surgical tool.

In another aspect of the invention, in a surgical system, a force sensor indicates (measures) an approximate force and/or moment at the first and/or the second end of the holding arm during the surgical procedure. A controller includes a force predictor for predicting (calculating) a force and/or moment at said end of the holding arm while performing tasks during the surgical procedure. The force predictor calculates a theoretical force and/or moment. An immediate deactivator immediately deactivates the robot when the indicated force and/or moment deviates from the predicted force and/or moment according to a predetermined criteria for determining when the human operator manually manipulates the second end of the holding arm and/or the surgical tool.

In another aspect of the invention, in a surgical system, a force sensor indicates (measures) an approximate force and/or moment at the first and/or the second end of a holding arm during the surgical procedure. An initial force and/or moment at the end of the holding arm is determined when the robot is activated. An immediate deactivator immediately deactivates the robot when the difference between the indicated force and/or moment and the initial force and/or moment exceeds a threshold for determining when the human operator manually manipulates the second end of the holding arm and/or the surgical tool.

In another aspect of the invention, in a surgical system, a grasp sensitive switch is positioned at one or more of: the distal end of a holding arm and/or a surgical tool near the holding arm. An immediate deactivator immediately deactivates the robot when the grasp sensitive switch is triggered when the operator grasps the distal end of the holding arm and/or the exterior portion of the surgical tool.

In another aspect of the invention, in a surgical system, the system includes a flexibility adjuster (stiffener/destiffener) to increasing and decreasing the flexibility of a holding arm and the flexibility adjuster is manually controlled by a lever on the holding arm. The lever may also deactivate the robot when the lever is set to increase the flexibility of the holding arm and may also activate the robot when the lever is set to decrease the flexibility of the holding arm.

In another aspect of the invention, in a surgical system, a flexibility adjuster of a holding arm is operated automatically by the robot. When the robot is activated the robot causes the flexibility adjuster to increase the stiffness of the holding arm, and when the robot is deactivated the robot causes the flexibility means to decrease the stiffness of the holding arm. The stiffener/destiffener can operate mechanically, pneumatically and/or piezoelectrically.

In another aspect of the invention, in a surgical system, an immediate deactivator immediately deactivates a robot when a signal of a condition sensor a predetermined threshold or criteria to be exceeded and the threshold or criteria can be adjusted using a user input.

In another aspect of the invention, in a surgical system, the system includes a microphone for initiating preprogrammed tasks by verbal commands and a foot switch for activating the robot to switch from the inactive mode to the active mode.

In another aspect of the invention, in a surgical system, an immediate deactivating means deactivates the robot by shutting off all power to motors of the robot.

In another aspect of the invention, in a surgical system, a robot includes an active arm having an end connected to the first end of a passive holding arm.

In an aspect of the invention, a method of operating a surgical system, includes the following steps. In response to a first action of a human operator, a robot is switched from a inactive mode to an active mode of robot operation during a surgical procedure. The surgical system is operated with a robot in an active mode. The robot may be preprogrammed with predetermined tasks or guided by the surgeon using, for example, a joystick. The robot may include a user input for a user to initiate the execution of the tasks in the active mode, the initiated tasks being executed in the active mode of operation. The surgical system includes an elongate holding arm with a first end and a second distal end. The first end of the holding arm is connected to the robot and the second distal end of the holding arm is connected to a surgical tool. The robot controls the repositioning of the holding arm for controlling the repositioning of the surgical tool of the surgical system during a surgical procedure. The holding arm is sufficiently stiff in the active mode to allow the robot to apply sufficient forces and moments through the holding arm to the surgical tool to perform the tasks during the surgical procedure, the holding arm being entirely passive during the surgical procedure.

The method further includes the following steps: In response to the human operator manipulating the surgical tool and/or the distal end of the holding arm, the robot immediately switches from the active mode of robot operation into a inactive mode of robot operation, the robot being substantially immobile when in the inactive mode during the surgical procedure. While in the inactive mode, increasing the flexibility of the passive holding arm sufficiently to allow a human operator to skillfully control repositioning the surgical tool into a new position while the holding arm is connected between the immobile robot and the surgical tool. Also, while in the inactive mode, decreasing the flexibility of the passive holding arm (130) sufficiently for the robot (100) to apply sufficient forces and moments through the holding arm (130) to the surgical tool (105) to perform the tasks in the active mode during the surgical procedure.

In endoscopic robotics it is important to make robot-surgeon interaction as close as possible to standard clinical practice (without the robot). Using head-mounted sensors might bring discomfort to the surgeon and might be less reliable if IR sensors are used and the light-of-sight gets disrupted in the operating room. Also, speech control of the robot might not work properly since it is difficult to pre-program all possible combinations of movements. Also, in moments of emergency, a surgeon inexperienced in the particular robot architecture, might, under stress, forget to press foot pedal or forget a verbal command and thus fail to take over the control over the robot.

Additional objects, features and advantages of the various aspects of the invention herein will become apparent from the following description in conjunction with the following drawings:

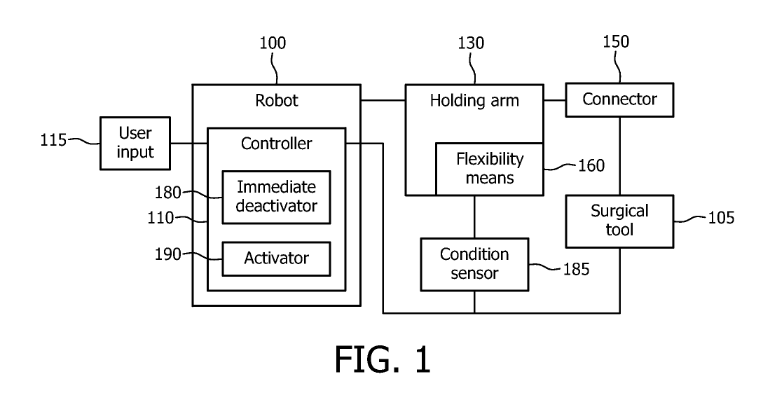

FIG. 1 is a schematic illustration of portions of the surgical system of the invention.

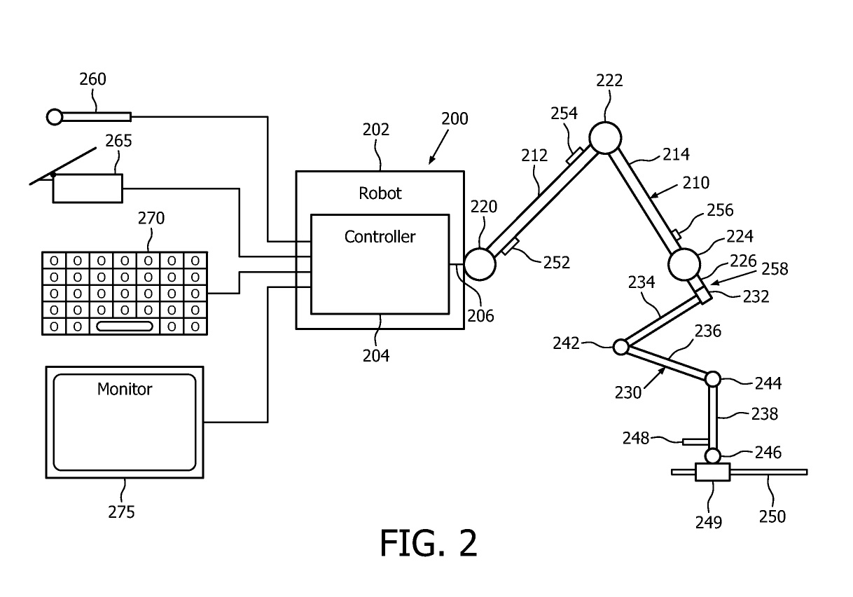

FIG. 2 shows a specific embodiment of portions of the holding arm and surgical tool of FIG. 1.

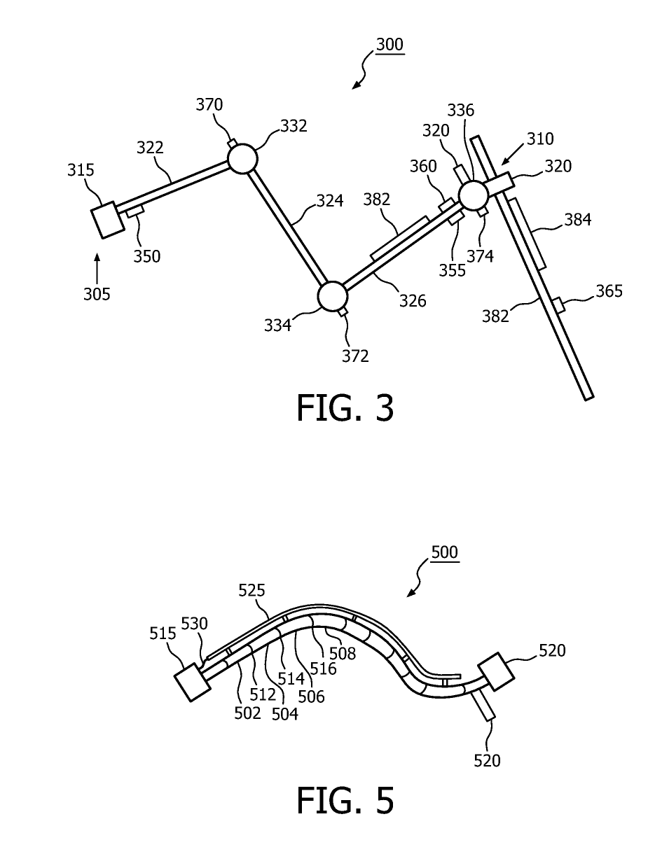

FIG. 3 illustrates another specific embodiment of portions of the holding arm of FIG. 1.

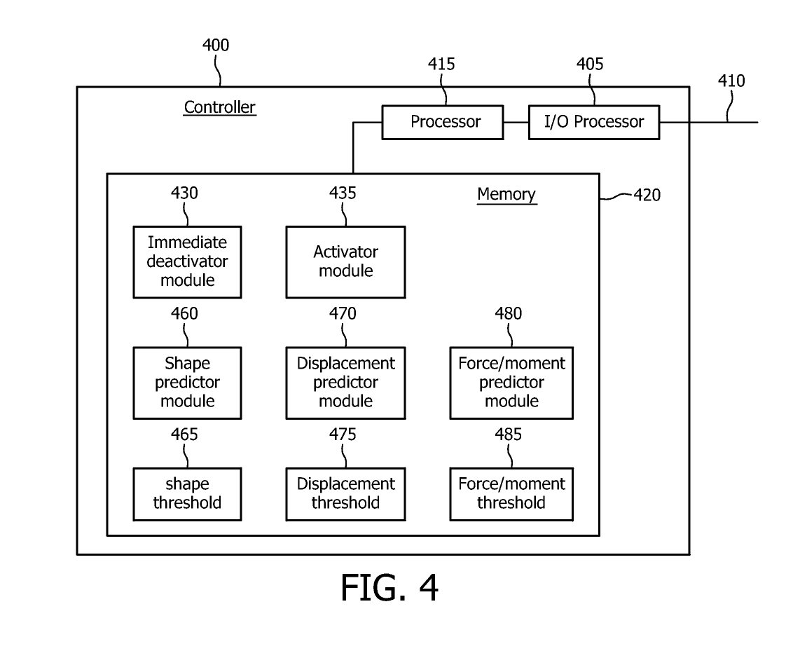

FIG. 4 schematically illustrates a specific embodiment of portions of a controller of the invention of FIG. 1.

FIG. 5 is a schematic of an example embodiment of portions of the surgical system of FIG. 1.

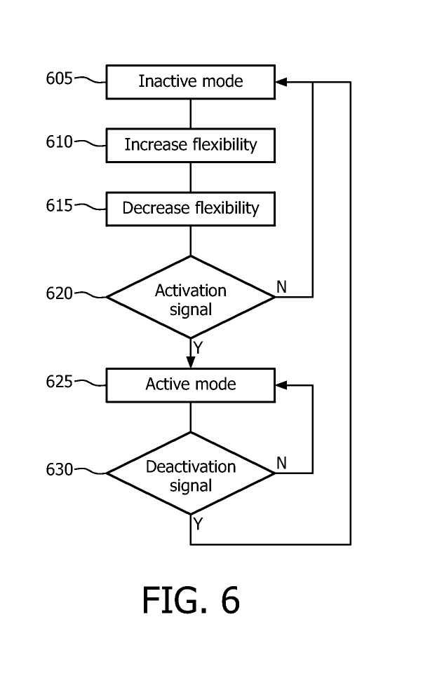

FIG. 6 is a flow diagram illustrating a specific embodiment of a portion of the operation of the surgical system of FIG. 1.

This invention proposes a method to simplify robot-surgeon interaction in endoscopy by allowing the robot to perform tasks, but also allowing the surgeon to instantly take manual control over the endoscope and allowing surgeon to reactivate robotic control subsequently. If the surgeon grasps the surgical tool and/or the robot arm and/or a passive holding arm at the surgical tool and/or otherwise attempts to manually manipulate the surgical tool, then the robot immediately goes into an inactive mode of operation. Means are provided to reduce the stiffness of the system when the robot is inactive to allow the surgeon to manually move the surgical tool in a manner similar to manual surgery. Means are also provided to increase the stiffness of the system after the manual manipulation is complete so that after reactivation the robot can perform further automated tasks in the active mode.

The specific embodiments will now be described with reference to the figures. Reference numbers beginning with 1 refer to FIG. 1, and reference numbers beginning with 2 refer to FIG. 2, and reference numbers beginning with 3 refer to FIG. 3, and reference numbers beginning with 4 refer to FIG. 4, and reference numbers beginning with 5 refer to FIG. 5, and reference numbers beginning with 6 refer to FIG. 6.

FIG. 1 is a schematic illustration of some portions of the surgical system of the invention. In FIG. 1, the surgical system, includes a robot (100) with both an active mode of operation and an inactive mode of operation. In the active mode the robot controls the repositioning of a surgical tool (105) during a surgical procedure. In the inactive mode the robot (100) is substantially immobile. The robot can be any mechanism adapted to move the surgical tool (105) during a surgical procedure. The robot may provide any number of degrees of freedom such as 3 degrees-of-freedom (DOF), 5 DOF or 6 DOF.

The robot (100) includes a controller (110) preprogrammed with predetermined tasks. The controller can be any means to control the robot to perform surgical tasks during a surgical procedure. The controller can be implemented purely in hardware or it may include programmed modules in a memory that control a processor as described below with respect to a specific embodiment illustrated in FIG. 4. The controller may include several interrelated controllers of a single central controller.

The surgical system of FIG. 1 also includes user input (115) communicating with the controller (110) for a user to initiate the execution of the preprogrammed tasks in the active mode. The user input may include a microphone and voice recognition module for verbal initiation of tasks, a foot pedal for activating the robot, and/or a keyboard for non-verbal initiation of tasks. The input may also include such itemed as push buttons, a mouse, a joystick, a track ball, a head-mounted pointer or any other user input device.

The surgical system also utilizes an elongate holding arm (130) with a first end connected to the robot and a second distal end having a connector (150) for connection to a removable surgical tool (105). The surgical tool may be, for example, an endoscope, a scalpel, a shaver, a pincher, a laser scalpel or any other common tool used in robotic surgery.

The holding arm (130) includes some means for flexibility adjustment (160) (stiffener/destiffener) for increasing or reducing the flexibility of the holding arm. Flexibility adjustment (160) may be used to increase the flexibility of the holding arm (130) for providing sufficient flexibility in an inactive mode to allow a human operator to skillfully control repositioning the surgical tool (105) into a new position while the flexible holding arm (130) is connected between the robot (100) and the surgical tool (105). Also, the flexibility adjustment, may be used for decreasing the flexibility of the holding arm (130) for locking it into a rigid fixed shape for providing sufficient rigidity in the active mode for the robot (100) to reposition the rigid holding arm (130) for repositioning the surgical tool (105). Snake-like arms with flexibility adjustment are well known, for example, FlexArm (Mediflex Inc. Canada). The stiffener/destiffener (160) can be operated by mechanical, pneumatic or piezoelectric means.

The surgical system also includes at least one condition sensor (185) that communicates with the controller (110), for producing signals depending on a mechanical condition of the holding arm (130) or surgical tool (105). The condition sensor (185) may be a shape sensor that may be connected along the length of the holding arm to signal the shape of the holding arm. Longate shape sensors are well known, such as, ShapeTape by (Measurand Inc. Canada) or Bragg grated fibers such as OBR Platform (Lune Technologies). The condition sensor (185) may be a position sensor such as an optical tracking or electromagnetic tracking device connected at the distal end of the holding arm or somewhere along the surgical tool. Optical and electromagnetic tracking devices are available from NDI (Northern Digital Inc.). The condition sensor (185) may be a force and/or moment sensor at either end of the holding arm (130) and/or on the surgical tool (105), such as, a strain gauge or load cell. Also, the condition sensor (185) may be a grasp sensing switch along the surgical tool and the distal end of the holding arm that produces a signal whenever the user grasps the surgical tool (105) and/or the distal end of the holding arm (130). A grasp sensor is different than a push-button because merely touching the holding arm (130) and/or surgical tool on a grasp sensor would not produce a signal indicating that the holding arm and/or surgical tool had been grasped, but it would be necessary to actually grasp the holding arm (130) or surgical tool for the grasp sensor signal to indicate that the holding arm or surgical tool had been grasped. Multiple condition sensors of the same and/or different types may be provided.

The surgical system also includes immediate deactivator (180) that determines when a human operator manually manipulates the holding arm (130) and/or the surgical tool (105) depending on signals from the condition sensor (185). When its determined that the human operator has manually manipulated the surgical tool (105) and/or the second end of the holding arm (130), then the immediate deactivator immediately deactivates the robot (100), by changing the mode of operation of the robot (100) from active mode to inactive mode.

The immediate deactivator (180) may be implemented as a programmed module in a memory of a controller which module controls the operation of a processor. Otherwise, the immediate deactivator (180) it may be implemented in hardware connected to control the operation of the processor. It may be part of the controller (100) of the robot, as shown, or it may be implemented as part of a separate deactivation controller as discussed below with respect to FIG. 4.

The immediate deactivator (180) may deactivate the robot by turning off all power to the robot motors. The depowering of the motors may be used to freeze the robot into a safe mode. If the robot motors are not a type of motor that freezes when power is cut off then the motors may be equipped with brakes that freeze the motors.

The surgical system of FIG. 1 also includes activator (190) that activates or reactivates the robot (100) in response to a signal from user input (115) by changing the mode of operation from inactive mode to active mode in the current position of the surgical tool (105). That is, the robot takes control of the robot arm and holding arm and surgical tool in the current position rather than returning the robot arm or the surgical tool to a previous position. When the robot (100) is activated it resumes controlling the repositioning of the surgical tool (105) during the surgical procedure. That is, it resumes executing the preprogrammed tasks initiated by the user utilizing the user input (115). For example, when the robot is in the inactive mode, then a foot switch may be used to activate the robot. The activator (180) may be implemented as a programmed module in a memory of a controller that controls the operation of a processor, or it may be implemented in hardware connected to control the operation of the processor. It may be part of the controller (100) of the robot, as shown, or it may be implemented as part of a separate activation controller as discussed below with respect to FIG. 4.

FIG. 2 is a schematic of an example embodiment of portions of the surgical system of FIG. 1. In FIG. 2, A robot indicated by arrow (200) includes a robot body/cabinet (202) containing controller (204), and also a robot arm indicated by arrow (210). The robot arm includes two segments (212, 214) connected by three motorized joints (220, 222, 224). The third joint (224) is an end effecter for positioning connector (226) for connection of a holding arm (230). A cable (206) connects between the controller (204) and electrical/electronic components of holding arm (230) such as joint motors (220, 222, 224) and sensors (shown below in relation to FIG. 3).

In FIG. 2, holding arm (230) includes a connector (232) to connect to the connector (226) of the robot arm. The holding arm includes three segments (234, 236, 238) connected together by three joints (242, 244, 246). Lever (548) can be used to adjust the stiffness of the joints between a very flexible setting in which the arm is easily manipulated and a rigid setting in which the arm is relatively rigid. Connector (249) is attached to joint (246) and is used for connecting a surgical tool (250) to the holding arm (230).

A microphone (260) may be connected to the controller for user input of voice commands. The verbal commands may include commands to initiate tasks that the robot is preprogrammed to perform, for example, to assist in a surgical procedure. The microphone may also be used to activate the robot or deactivate the robot. Also, voice commands may be used to adjust the flexibility of the holding arm between a very flexible state and a rigid state.

A foot switch (265) is connected to the controller for a user signal. The signal may be a signal to initiate robot activation. Activation of the robot may also cause the flexibility means (160) to cause the holding arm to become rigid.

A keyboard (270) is also connected to the controller for non-audio input of commands. The commands may be any of the commands discussed above in relation to microphone (260).

A visual output device such as a monitor is connected to the controller for providing the user with status information. For example, when the user uses the microphone to make a verbal command, then the command is shown on the monitor.

Other input devices such as a mouse or joystick or trackball or head mounted pointer or gloves may be provided for command input.

The robot arm (210) may include one or more condition sensors (184) (in FIG. 1). As shown in FIG. 2, the sensors (252, 254, 256) may be, for example, force/moment sensor that signals the force and/or moments on the connectors or joints of the robot arm during a surgical procedure. The sensors (252, 254, 256) may be tracking sensors to indicate the position of the end (258) of the robot arm during a surgical procedure. The sensors (252, 254, 256) may be position sensors to indicate the positions of the joints of the holding arm during a surgical procedure. The sensor (256) may be a grasp sensor that detects when someone grasps near the end (258) of the robot arm.

FIG. 3 is a specific embodiment of portions of the holding arm (130) and surgical tool (105) of FIG. 1. In FIG. 3, the holding arm (300) is a elongate structure having a first end (305) and a second distal end (310). The first end (305) of the holding arm has a connector (315) for connection to the robot (100) (in FIG. 1), and in FIG. 3, the second end (310) of the holding arm (300) has a connector (320) for connecting a surgical tool (302) to the distal end of the holding arm. In general, it is expected that the holding arm will have more degrees-of-freedom than the robot arm. The holding arm (300) comprises multiple arm segments (322, 324, 326) connected together by multiple joints (332, 334, 336). The holding arm (300) is purely inactive having no means for self motion. The robot (100) will move the first end of the holding arm to move the second end of the holding arm to move the surgical tool/instrument.

A lever (320) on the holding arm (300) can be used to manually adjust the flexibility of the arm by adjusting the force/moment required to rotate the joints. Alternatively, or in addition, the flexibility of the joints of the holding arm may be adjusted by the robot using connection (315) to the robot. In a stiff setting of the flexibility means, the joints are sufficiently rigid so that the joints will not rotate when the robot is in an active mode performing tasks during a surgical procedure. The holding arm may be very stiff or locked so that the joints are essentially frozen. In a flexible setting, the stiffness of the holding arm is sufficiently flexible so that a surgeon, assistant or other user can manually manipulate a surgical tool (302) during a surgical procedure to change the position of the surgical tool (302). In the flexible setting the holding arm is sufficiently stiff so that the surgical tool will not move unless manipulated by the user.

The immediate deactivator (180) in FIG. 1 may immediately deactivate the robot (100) when the flexibility means is activated to increase the flexibility of the holding arm. For example, in FIG. 3, lever (320) can be connected to the controller through a motion transducer, so that, the immediate deactivator initiates to deactivate the robot when the lever is turned for increasing the flexibility of the holding arm. Similarly, the immediate deactivator may operate the flexibility means so that when the robot is deactivated it causes the flexibility means to reduce the stiffness of the holding arm. Also, activating the robot may cause the flexibility means to increase the stiffness of the holding arm sufficient for performing tasks during the surgical procedure.

The holding arm (300) includes one or more condition sensors (184) (in FIG. 1). As shown in FIG. 3, the sensors may include force/moment sensors (350, 355) on the robot arm and/or holding arm that signals the force and/or moments on the connectors or joints of the holding arm during a surgical procedure. The sensors may also include a tracking sensors (360, 365) to indicate the position of the surgical tool (302) or the distal end (310) of the holding arm (300) during a surgical procedure. The sensors may include position sensors (370, 372, 374) to indicate the positions of the joints of the holding arm during a surgical procedure. The sensors may include grasp sensors (382, 384) that detect when someone grasps the surgical tool (302) and/or the distal end of the holding arm.

FIG. 4 schematically illustrates a specific embodiment of portions of a controller (400) of the invention. I/O processor (405) is connected to an I/O bus (410) to provide signals and to receive signals through the bus. The input signals may include signals from at least one condition sensor (185) (in FIG. 1) and signals from user input (115) (in FIG. 1) and output signals may include signals to control motors of the robot (100) (in FIG. 1). I/O processor (405) is connected to processor (415) which is a CPU, embedded processor, or general processor. CPU (415) is controlled by program modules stored in memory (420).

The modules of memory (420) include an immediate deactivator module (430) to implement the immediate deactivator (180) (in FIG. 1). In FIG. 4, when signals from the condition sensor (185) (in FIG. 1) are detected then the immediate deactivator module (430) controls the CPU to determine whether the user is manipulating the surgical tool and/or the distal end of the holding arm, and if so, then the immediate deactivator (430) immediately deactivates the robot. This specific embodiment also includes an activator module (435) to implement the activator (190) (in FIG. 1). In FIG. 4, when the user signals the activation, for example, using a foot switch, then the activator module determines if the robot should be activated, and if its determined to activate the robot, then the activator module activates the robot.

In a specific embodiment of condition sensor 180 (in FIG. 1), a shape sensor (525) (in FIG. 5) on the holding arm (500) (in FIG. 5) indicates the approximate shape of the holding arm during the surgical procedure. In FIG. 4, shape predicting module (460) predicts the shape of the holding arm while tasks are performed during the surgical procedure. The immediate deactivation module (430) deactivates the robot (100) (in FIG. 1) when the approximate shape deviates from the predicted shape according to a predetermined criteria for determining when the human operator manually manipulates the second end of the holding arm and/or the surgical tool. The predetermined criteria may, for example, be a threshold for the deviation or may include other criteria that may be related to other condition sensors of the surgical system as described below.

Alternatively or in addition, an initial shape of the flexible arm is determined when the robot (100) (in FIG. 1) is activated, and in FIG. 4, the immediate deactivation module (430) deactivates the robot when the difference between the indicated shape and the initial shape exceeds a threshold (465) for determining when the human operator is manually manipulating the second end of the holding arm and/or the surgical tool.

In another specific embodiment of condition sensor 180 (in FIG. 1), a displacement sensor (360,365) (in FIG. 3) indicates an approximate linear and/or rotational displacement of the surgical tool (382) (in FIG. 3) and/or the distal end (310) (in FIG. 3) of the holding arm during the surgical procedure. Typically this function is performed using a tracking sensor. In FIG. 4, displacement predicting module (470) predicts the linear and/or rotational displacements of the surgical tool and/or the distal end of the holding arm while performing tasks during the surgical procedure. The immediate deactivation module (430) deactivates the robot (100) (in FIG. 1) when the indicated displacement deviates from the predicted displacement according to a predetermined criteria for determining when the human operator manually manipulates the second end of the holding arm and/or the surgical tool. The predetermined criteria can be a threshold for the deviation or may include other criteria related to other condition sensors of the surgical system as described below.

Alternatively or in addition, an initial linear and/or rotational displacement of the surgical tool (382) (in FIG. 3) and/or the distal end (310) (in FIG. 3) of the holding arm is determined when the robot (100) (in FIG. 1) is activated. In FIG. 4, he immediate deactivating module (430) deactivates the robot when the difference between the linear and/or rotational displacement and the initial linear and/or rotational displacement exceeds a threshold (475) for determining that the human operator is manually manipulating the second end of the holding arm and/or the surgical tool.

In another specific embodiment of condition sensor (180) (in FIG. 1), a force sensor (350,355) (in FIG. 3) indicates an approximate force and/or moment at the first and/or second end of the holding arm (300) (in FIG. 3) during the surgical procedure. In FIG. 4, controller (400) includes force predicting module (480) for predicting a force and/or moment at said end of the holding arm while performing tasks during the surgical procedure. Immediate deactivation module (430) deactivates the robot (100) (in FIG. 1) when the indicated force and/or moment deviates from the predicted force and/or moment according to a predetermined criteria for determining when the human operator manually manipulates the second end of the holding arm and/or the surgical tool. The predetermined criteria can be a threshold for the deviation or may include other criteria related to other condition sensors of the surgical system as described below.

Alternatively or in addition, in FIG. 1 an initial force and/or moment at the first and/or second end of the holding arm (130) is determined when the robot (100) is activated. In FIG. 4, the immediate deactivation module (430) deactivates the robot when the difference between the indicated force and/or moment and the initial force and/or moment exceeds a threshold (485) for determining when the human operator manually manipulates the second end of the holding arm and/or the surgical tool.

The thresholds (465, 475, 485) may be adjusted using user input (115) (in FIG. 1). For example, the thresholds may need to be higher during some surgical procedures and lower in other surgical procedures, or some users may want higher thresholds and other users may want lower thresholds.

Also, in FIG. 3, a grasp sensitive switch (382, 384) is positioned at one or more of: the distal end of the holding arm (130) (in FIG. 1) or the surgical tool (105) near the holding arm. The immediate deactivation module (430) (in FIG. 4) deactivates the robot (100) (in FIG. 1) when the when the grasp sensitive switch is activated by the operator grasping the distal end of the holding arm and/or the surgical tool. A grasp sensitive sensor is distinguished from a push button because merely pushing the grasp sensitive sensor with a finger does not initiate a signal, on the contrary, a signal will only be generated by grasping the object to which the grasp sensitive sensor is attached (the surgical tool and/or the holding arm).

The predetermined criteria that initiates the immediate deactivation of robot may be a combined criteria, such as, it may be required that both the deviation of the shape of the holding arm exceeds a threshold and that the deviation of the force/moment at a joint of the holding arm exceeds a threshold.

FIG. 5 illustrates an alternative embodiment of the holding arm (500) of the invention. In FIG. 5 a snake-like holding arm (500) comprises a multitude of segments (502, 504, 506, 508) connected together by a multitude of joints (512, 514, 516). A lever (470) is connected to all the joints of the holding arm by internal wires to adjust the stiffness of the holding arm. The holding arm includes a elongate shape sensor (525) to indicate the approximate shape of the holding arm during a surgical procedure. The shape sensor is connected along its length of the holding arm. A signal conductor (530) is routed through connector (515) to the controller (110) The shape sensor can be, for example, shape tape or Bragg grated fibers or other types of shape sensors as discussed above for condition sensor (185) in FIG. 1.

FIG. 6 is a flow diagram illustrating a specific embodiment of a portion of the operation of the surgical system of FIG. 1. The figure only illustrates the operations related to transitions between inactive mode and active mode. The flow diagram does not illustrate initial startup of final shutdown of the surgical system. In step (605), the flow chart begins with the robot in the inactive mode. In the inactive mode the motors of the robot (100) are shut down. They may be shut down by cutting off all power to the motors and/or motor breaks/locks may be provided. The robot is rigid and immobile so that the robot will not accidentally move during the surgical procedure.

In step (610), while in the inactive mode, the flexibility of holding arm (130) may be increased sufficiently to allow the surgical tool (105) and/or holding arm (130) to be manipulated so that a surgical tool is manually repositioned by the user. The increased flexibility may be provided such that the surgical tool would move without the user applying force to move it. The flexibility may be increased manually and/or the flexibility may be increased automatically by the robot (100) being switched into the inactive mode.

In step (615) while in the inactive mode, the flexibility of the holding arm (130) can be decreased sufficiently to allow the robot to control the movement of the surgical tool (105) during surgical tasks. The holding arm may be made essentially rigid and substantially inflexible. The flexibility may be decreased manually. When the holding arm is manually made flexible, then the holding arm should be made rigid before the robot is switched into the active mode. Also, the flexibility may be decreased automatically by the robot (100) being switched into the active mode in step (625) described below.

While in the inactive mode, at step (620), the surgical system continually scans for an activation signal to activate the robot. If there is no activation signal then the robot continues to operate in the inactive mode. if there is an activation signal then the robot switches into the active mode as described below. The activation signal may be provided by a foot switch or a simple push button on the robot (100) or on the holding arm (130).

In step (625) the robot operates in the active mode. The robot is preprogrammed with predetermined tasks. The robot including user input means (115) for a user to initiate the execution of the tasks. The surgical system includes an elongate holding arm (130) with a first end (305) and a second distal end (310), the first end (305) of the holding arm being connected to the robot (100) and the second distal end (310) of the holding arm being connected to a surgical tool (105). In the active mode, the robot (100) controls the repositioning of the holding arm (130) for controlling the repositioning of the surgical tool (105) of the surgical system during a surgical procedure. The holding arm (130) is sufficiently stiff in the active mode to allow the robot (100) to apply sufficient forces and moments through the holding arm (130) to the surgical tool (105) to perform the tasks during the surgical procedure. The holding arm (130) has no motors or other means for self-movement and thus it remains entirely passive during the surgical procedure.

While in the active mode, at step (630), the surgical system continually scans for a deactivation signal to deactivate the robot. Sensors are provided on the robot arm (210) and/or the holding arm (130) and/or the surgical tool (105) for indicating when the user is attempting to manually manipulate the surgical tool (105) and/or the second end of the holding arm (130). An immediate deactivator (180) uses a criteria to determine when the user is attempting to manually manipulate the surgical tool (105) and/or the second end of the holding arm (130). Upon said determination, then the robot is immediately deactivated, by changing the mode of operation of the robot (100) from active mode to inactive mode.

Finally, the above-discussion is intended to be merely illustrative of the present invention and should not be construed as limiting the appended claims to any particular embodiment or group of embodiments. Each of the systems utilized may also be utilized in conjunction with further systems. Thus, while the present invention has been described in particular detail with reference to specific exemplary embodiments thereof, it should also be appreciated that numerous modifications and changes may be made thereto without departing from the broader and intended spirit and scope of the invention as set forth in the claims that follow. The specification and drawings are accordingly to be regarded in an illustrative manner and are not intended to limit the scope of the appended claims.

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference numerals in the claims are for illustration purposes only and do not limit their protective scope;
d) several “means” may be represented by the same item or hardware or software implemented structure or function; and
e) each of the disclosed elements may be comprised of hardware portions (e.g., discrete electronic circuitry), software portions (e.g., computer programming), or any combination thereof.

1. A surgical system, comprising:
a robot (100) with an active mode of operation for controlling the repositioning of a surgical tool (105) during a surgical procedure, and an inactive mode of operation in which the robot (100) is substantially immobile, the robot (100) having control means (110) preprogrammed with predetermined tasks to be performed during a surgical procedure;
a user input (115) communicating with the control means (110) for a user to initiate the execution of the preprogrammed tasks in the active mode;
an elongate holding arm (130) with a first end (305) and a second distal end (310), the first end having a connector (315) for connection to the robot (100), the second distal end having a connector (150) for connection to the surgical tool (105);
the holding arm (130) including flexibility means (160) for increasing the flexibility of the holding arm (130) for providing sufficient flexibility in an inactive mode to allow a human operator to skillfully control repositioning the surgical tool (105) into a new position while the flexible holding arm (130) is connected between the robot (100) and the surgical tool (105), and for decreasing the flexibility of the holding arm (130) for locking it into a rigid fixed shape for providing sufficient rigidity in the active mode for the robot (100) to reposition the rigid holding arm (130) for repositioning the surgical tool (105) to perform the tasks;
a condition sensor (185) communicating with the control means(110) for producing signals depending on a mechanical condition of the holding arm (130) or surgical tool (105);
immediate deactivation means (180) for determining when a human operator manually manipulates the holding arm (130) or the surgical tool (105) depending on signals from the condition sensor (185); and for immediately deactivating the robot (100) by changing the mode of operation of the robot (100) from active mode to inactive mode when its determined that the human operator manually manipulates the second end of the holding arm (130) or the surgical tool (105);
activation means (190) for activating the robot (100) in response to user input means (115) by changing the mode of operation of the robot from inactive mode to active mode in a current position of the surgical tool (105) and for the robot (100) to resume controlling the repositioning of the surgical tool (105) during the surgical procedure.

2. The surgical system of claim 1, wherein the condition sensor (185) include shape sensors (575) on the holding arm (130) for indicating the approximate shape of the holding arm (130) during the surgical procedure, and the control means (110) includes shape predicting means (460) for predicting the shapes of the holding arm while performing tasks during the surgical procedure, and the immediate deactivation means (180) deactivates the robot when the indicated shape deviates from the predicted shape according to a predetermined criteria for determining when the human operator manually manipulates the second end of the holding arm or the surgical tool.

3. The surgical system of claim 1, wherein the condition sensors (185) include shape sensors (310) on the holding arm (130) for indicating the approximate shape of the holding arm (130) during the surgical procedure, and an initial shape of the flexible arm is determined when the robot is activated, and the immediate deactivation means (180) deactivates the robot (100) when the difference between the indicated shape and the initial shape exceeds a threshold (465) for determining when the human operator is manually manipulating the second end of the holding arm or the surgical tool.

4. The surgical system of claim 1, wherein the condition sensor (185) includes a displacement sensor (320) for indicating an approximate linear or rotational displacement of the surgical tool (105) or the distal end of the holding arm (130) during the surgical procedure; and the control means (110) includes displacement predicting means (470) for predicting linear or rotational displacements of the surgical tool (105) or the distal end of the holding arm (130) while performing tasks during the surgical procedure, and the immediate deactivation means (180) deactivates the robot (100) when the indicated displacement deviates from the predicted displacement according to a predetermined criteria for determining when the human operator manually manipulates the second end of the holding arm or the surgical tool.

5. The surgical system of claim 1 wherein the displacement sensor (320) is an electromagnetic or optical displacement sensor.

6. The surgical system of claim 1 wherein the condition sensor (185) includes a displacement sensor (320) for indicating an approximate linear or rotational displacement of the surgical tool (105) or the distal end of the holding arm (130) during the surgical procedure; and an initial linear or rotational displacement of the surgical tool (105) or the distal end of the holding arm (130) is determined when the robot is activated, and the immediate deactivating means (180) deactivates the robot (100) when the difference between the linear or rotational displacement and the initial linear or rotational displacement exceeds a threshold (475) for determining that the human operator is manually manipulating the second end of the holding arm or the surgical tool.

7. The surgical system of claim 1, wherein the condition sensor (185) includes a force sensor (330) for indicating an approximate force or moment at the first or the second end of the holding arm (130) during the surgical procedure; and the control means (110) includes force predicting means (480) for predicting a force or moment at said end of the holding arm (130) while performing tasks during the surgical procedure, and the immediate deactivation means (180) deactivates the robot (100) when the indicated force or moment deviates from the predicted force or moment according to a predetermined criteria for determining when the human operator manually manipulates the second end of the holding arm or the surgical tool.

8. The surgical system of claim 1, wherein the condition sensor (185) includes a force sensor (330) for indicating an approximate force or moment at the first or the second end of the holding arm (130) during the surgical procedure; and an initial force or moment at the end of the holding arm is determined when the robot is activated, and the immediate deactivation means (180) deactivates the robot (100) when the difference between the indicated force or moment and the initial force or moment exceeds a threshold (485) for determining when the human operator manually manipulates the second end of the holding arm or the surgical tool.

9. The surgical system of claim 1, comprising a grasp sensitive switch (340,450) positioned at one or more of: the distal end of the holding arm (130) or the surgical tool (105) near the holding arm, and the immediate deactivation means (180) deactivates the robot (100) when the grasp sensitive switch is triggered when the operator grasps the distal end of the holding arm or a grasping end of the surgical tool.

10. The surgical system of claim 1, wherein the flexibility means (160) comprises a flexibility lever (320) on the holding arm to manually adjust the flexibility of the holding arm (130).

11. The surgical system of claim 9, wherein the controller (110) deactivates the robot (100) when the flexibility lever (320) is used to increase the flexibility of the holding arm (130).

12. The surgical system of claim 1 wherein activating the robot (110) causes the flexibility means to increase the stiffness of the holding arm and deactivating the robot causes the flexibility means to decrease the stiffness of the holding arm (130).

13. The surgical system of claim 1, wherein the immediate deactivation means deactivates the robot when the signal of a condition sensor causes a predetermined threshold or criteria to be exceeded and the threshold or criteria can be adjusted using user input means (115).

14. The surgical system of claim 1, comprising a surgical tool and the surgical tool is an endoscope.

15. The surgical system of claim 1 wherein the user input means (115) includes a microphone (260) and the execution of at least one of the preprogrammed tasks can be initiated by verbal commands detected by the microphone (260).

16. The surgical system of claim 1 wherein user input means (115) includes a foot switch (265) and the means for reactivating the robot to switch from the inactive mode to the active mode is initiated by the foot switch (265).

17. The surgical system of claim 1, wherein the immediate deactivating means (180) deactivates the robot (100) by shutting off all power to motors of the robot (100).

18. A method of operating a surgical system, the method comprising:
in response to a user input, switching a robot (100) from an inactive mode to an active mode of robot operation during a surgical procedure;
operating the surgical system with a robot in an active mode, the robot being preprogrammed with predetermined tasks, the robot including user input means (115) for a user to initiate the execution of the tasks in the active mode, the initiated tasks being executed in the active mode of operation, the surgical system including an elongate holding arm (130) with a first end (335) and a second distal end (340), the first end (335) of the holding arm being connected to the robot (100) and the second distal end (340) of the holding arm being connected to a surgical tool (105), the robot (100) controlling the repositioning of the holding arm (130) for controlling the repositioning of the surgical tool (105) of the surgical system during a surgical procedure, the holding arm (130) being sufficiently stiff in the active mode to allow the robot (100) to apply sufficient forces and moments through the holding arm (130) to the surgical tool (105) to perform the tasks during the surgical procedure, the holding arm (130) being entirely inactive during the surgical procedure,
in response to the human operator manipulating the surgical tool (105) or the distal end of the holding arm (130), immediately switching from the active mode of robot operation into the inactive mode of robot operation; the robot being substantially immobile when in the inactive mode during the surgical procedure;
while in the inactive mode, increasing the flexibility of the inactive holding arm (130) sufficient for a human operator to skillfully control repositioning the surgical tool into a new position while the holding arm (130) is connected between the immobile robot (100) and the surgical tool (105);
while in the inactive mode, decreasing the flexibility of the inactive holding arm (130) sufficient for the robot (100) to apply sufficient forces and moments through the holding arm (130) to the surgical tool (105) to perform the tasks in the active mode during the surgical procedure.
